(19) United States
(12) Patent Application Publication
(10) Pub. No.: US 2010/0222282 A1
(43) Pub. Date: Sep. 2, 2010

(54) METHOD OF DIAGNOSIS AND METHOD OF TREATMENT

(75) Inventor: Stefan G. Pierzynowski, Malmo (SE)

Correspondence Address: SCHWEGMAN, LUNDBERG & WOESSNER, P.A., P.O. BOX 2938, MINNEAPOLIS, MN, 55402, US

(73) Assignee: ENTRESS AB, Stockholm (SE)

(21) Appl. No.: 12/161,538

(22) PCT Filed: Jan. 18, 2007
(86) PCT No.: PCT/EP2007/050499

§ 371 Date: Jul. 18, 2008

(30) Foreign Application Priority Data
Jan. 19, 2006 (SE) 0600130-9

(51) Int. Cl. A61K 38/06; A61K 31/191; A61K 31/195; A61K 38/05; A61K 31/722; C12Q 1/02; A61P 13/12

(52) U.S. Cl. 514/18; 514/574; 514/563; 514/19; 514/55; 435/29

(57) ABSTRACT

The present invention relates to a method for diagnosing higher susceptibility for diseases and conditions associated with low levels of AKG in a human or animal comprising the following steps: a) obtaining a biological sample from said human or animal; b) measuring the alpha-ketoglutaric acid (AKG) level in the biological sample; and c) comparing said measured AKG level with normal average AKG levels, wherein a level of AKG in said sample lower than an average level is indicative of a higher susceptibility for various diseases. Further the invention relates to a use of a substance comprising at least one member selected from the group consisting of AKG and derivates, metabolites, analogues or salts thereof for the manufacture of a pharmaceutical preparation or a food or feed supplement for the treatment or prophylaxis of diseases and conditions associated with low levels of AKG in a human or animal with low levels of AKG compared with normal average AKG levels.

### FIELD OF INVENTION

The present invention relates to methods of diagnosis, new use of known pharmacologically active chemical compounds, and methods of treatment or prophylaxis. More particularly, the present invention relates to methods for diagnosing higher susceptibility for various diseases and conditions in a human or animal; the new use of a certain acid and derivates, metabolites, analogues or salts thereof for the manufacture of a pharmaceutical preparation or a food or feed supplement for the in vivo therapeutic treatment or prophylaxis; and methods of treatment or prophylaxis of various diseases and conditions in a human or animal.

### BACKGROUND OF THE INVENTION

The morbidity and mortality of diseases or conditions associated with increased plasma levels of cholesterol, low density lipids and glycerides, such as arteriosclerosis, coronary disease, heart failure and cardiac death in developed countries is higher than that associated with any other disease or condition. It has been reported that about 50% of all deaths in the United States, Europe and Japan are due to atherosclerosis.

Osteoporosis, the increase in the brittleness of bones, is a health problem of aging people, particularly women, and in recent years this bone disease has been becoming a problem. Osteoporotic fractures and the complications associated with them have caused a significant increase in health care costs as the age structure of the population grows older.

Diseases or conditions associated with cartilage impairment are a common problem. Today approximately 14% of the present population of United States suffers from some type of arthritic manifestations.

Neoplastic diseases are one of the most significant health problems throughout the world, and the increased number of cancer cases reported around the world is a major concern.

It is estimated that diseases of the nervous system, such as Alzheimer's disease, currently affects over 2 million elderly people in the United States. Because the disorder is usually late onset, the number of affected individuals will continue to grow as the elderly population increases in size.

End-stage renal disease affects approximately 650 000 human patients each year worldwide and treatment costs have been estimated to be about twenty billion dollars.

Cirrhosis of the liver is a common condition that frequently goes undetected. For example, in a large sample of the general Danish population, the prevalence of liver cirrhosis was 4.5%, of which one-third were undiagnosed at the time of death.

Helicobacter pylori is estimated to be responsible for up to 90% of the cases of peptic ulcer disease (PUD) afflicts over 10% of the US population sometime in their lifetime. Estimates for worldwide prevalence of H. pylori infections range from 300 million to over two-thirds of the world's population. H. pylori infection is also associated with 650,000 annual cancer deaths worldwide from gastric adenocarcinoma.

There are today available a large number of diagnostic and therapeutic methods for the diseases and conditions mentioned above. Just to give some examples of these, the following documents can be mentioned: U.S. Pat. No. 5,731,208 discloses a screening test for atherosclerosis; U.S. Pat. No. 6,998,242 discloses a method of diagnosing metabolic bone diseases, especially osteoporosis and arthrosis; U.S. Pat. No. 4,402,934 discloses diagnosis of rheumatoid arthritis and related diseases; U.S. Pat. No. 6,682,901 discloses methods for diagnosing cancer; U.S. Pat. No. 6,391,553 discloses a method for diagnosing Alzheimer's disease; U.S. Pat. No. 7,045,282 discloses treatment and diagnosis of kidney diseases; U.S. Pat. No. 6,986,995 discloses methods of diagnosing liver diseases; and U.S. Pat. No. 7,014,612 discloses a method for diagnosis of Helicobacter pylori infection.

But still, at present, huge amounts of resources and efforts are invested to develop and improve diagnostic and therapeutic methods for the diseases and conditions mentioned above. New improved or more beneficial methods to treat and/or diagnose these above mentioned diseases and conditions could alleviate tremendous amount of human suffering and/or lower the today huge medical costs related to these diseases and conditions. It is, for example, desirable to develop diagnostic techniques which are easy, less costly and/or less time consuming to perform, or to provide early warning of the onset of the disease. Further, it is desirable to develop effective, safe, painless and convenient forms of treatment that can be employed to treat the diseases and conditions mentioned above.

### SUMMARY OF THE INVENTION

In accordance with the present invention, it was surprisingly found that a low level of AKG in a biological sample from a human or animal is indicative of a higher susceptibility for various diseases and conditions. Accordingly, discovering low levels of AKG in biological samples can be used not only for diagnostic and prognostic purposes for these various diseases and conditions but can also be used to direct therapeutic treatments or prophylaxis of these diseases and conditions.

According to a first aspect of the present invention, there is provided a method for diagnosing higher susceptibility for diseases and conditions associated with low levels of AKG in a human or animal comprising the following steps:

a) obtaining a biological sample from said human or animal;

b) measuring the alpha-ketoglutaric acid (AKG) level in the biological sample; and

c) comparing said measured AKG level with normal average AKG levels, wherein a level of AKG in said sample lower than said normal average levels is indicative of a higher susceptibility for diseases or conditions associated with low levels of AKG.

According to an embodiment of the invention, said diseases or conditions associated with low levels of AKG include: diseases or conditions associated with increased plasma levels of cholesterol, low density lipids and glycerides; diseases or conditions associated with bone loss or weakening; diseases or conditions associated with cartilage impairment; neoplastic diseases; diseases of the nervous system; renal failure; liver diseases; heart conditions; stroke; bad wound healing; and Helicobacter pylori related diseases or conditions of the gastrointestinal tract.

According to a further embodiment of the invention, said diseases or conditions associated with low levels of AKG include: arteriosclerosis, coronary disease, heart failure and cardiac death; osteoporosis; arthrosis and rheumatoid arthritis; neoplastic diseases and cancer; renal failure; liver cirrhosis, hepatitis or liver failure; heart attack; stroke; bad wound healing; Helicobacter pylori related gastritis, gastric and duodenal ulcers, peptic ulcer, gastric cancer, and gastric mucosa-associated lymphoid tissue lymphoma.

According to a further embodiment of the invention, the biological sample is blood or urine.

According to a further embodiment of the invention, in a human the normal average blood levels of AKG in a group of humans are in the range of 5-7 μg/ml.

According to a second aspect of the present invention, there is provided a use of a substance comprising at least one member selected from the group consisting of alpha-ketoglutaric acid (AKG) and derivates, metabolites, analogues or salts thereof for the manufacture of a pharmaceutical preparation or a food or feed supplement for the treatment of a human or animal with low levels of AKG compared with normal average AKG levels.

According to an embodiment of the invention, there is provided a use of a substance comprising at least one member is selected from the group consisting of alpha-ketoglutaric acid (AKG) and derivates, metabolites, analogues or salts thereof for the manufacture of a pharmaceutical preparation or a food or feed supplement for the treatment or prophylaxis of diseases and conditions associated with low levels of AKG in a human or animal with low levels of AKG compared with normal average AKG levels.

According to a further embodiment of the invention, said diseases and conditions associated with low levels of AKG include: diseases or conditions associated with increased plasma levels of cholesterol, low density lipids and glycerides; diseases or conditions associated with bone loss or weakening; diseases or conditions associated with cartilage impairment; neoplastic diseases; diseases of the nervous system; renal failure; liver diseases; heart conditions; stroke; bad wound healing; and Helicobacter pylori related diseases or conditions of the gastrointestinal tract.

According to another embodiment of the invention, said diseases or conditions associated with low levels of AKG include: arteriosclerosis, coronary disease, heart failure and cardiac death; osteoporosis; arthrosis and rheumatoid arthritis; neoplastic diseases and cancer; renal failure; liver cirrhosis, hepatitis or liver failure; heart attack; stroke; bad wound healing; Helicobacter pylori related gastritis, gastric and duodenal ulcers, peptic ulcer, gastric cancer, and gastric mucosa-associated lymphoid tissue lymphoma.

According to a further embodiment of the invention, said at least one member is selected from the group consisting of AKG, glutamine, glutamic acid and pharmaceutically acceptable salts of these acids, amides of AKG and an amino acid or a di- or tripeptide, di- or tripeptides of glutamine and other amino acids, di- or tripeptides of glutamic acid and other amino acids, pharmaceutically acceptable salts of said di- or tripeptides, and pharmaceutically accepted physical mixtures of AKG or a pharmaceutically acceptable salt thereof and at least one amino acid.

In a specific embodiment of the invention said at least one member is selected from the group consisting of ornithine-AKG, arginine-AKG, glutamine-AKG, glutamate-AKG, leucine-AKG, chitosan-AKG, an alkali or alkaline earth metal salt of AKG, or mixtures thereof.

According to a further embodiment of the invention the amount of AKG given to a human or animal is a therapeutically effective amount.

In specific embodiments the therapeutically effective amount is in the interval from 1 to 1000, 10 to 400, or 10 to 100 mg/kg body weight/day.

According to a further aspect of the present invention, there is provided a for the treatment or prophylaxis of diseases and conditions associated with low levels of AKG in a human or animal with low levels of AKG compared with normal average AKG levels, which method comprises administering to a subject in need for such treatment or prophylaxis of an effective amount of at least one member selected from the group consisting of alpha-ketoglutaric acid (AKG) and derivates, metabolites, analogues or salts thereof.

According to an embodiment of the invention, said diseases and conditions associated with low levels of AKG include: diseases or conditions associated with increased plasma levels of cholesterol, low density lipids and glycerides; diseases or conditions associated with bone loss or weakening; diseases or conditions associated with cartilage impairment; neoplastic diseases; diseases of the nervous system; renal failure; liver diseases; heart conditions; stroke; bad wound healing; and Helicobacter pylori related diseases or conditions of the gastrointestinal tract.

According to another aspect, the present invention encompasses a method of increasing quality of life comprising the following steps:

a) measuring the alpha-ketoglutaric acid (AKG) level in a biological sample;

b) supplementing a low level of AKG with AKG to normalize said AKG level.

These and other aspects and embodiments of the present invention will be described in more detail below.

### DETAILED DESCRIPTION OF THE INVENTION

The present invention is based on the inventor's knowledge and realization that levels of alpha-ketoglutaric acid (AKG) influence the general health condition of a human or animal. Studies conducted by the inventor have revealed that low levels of AKG in blood of a subject are indicative of a higher susceptibility for various diseases and conditions. Accordingly, discovering low levels of AKG in biological samples can be used not only for diagnostic and prognostic purposes but may also be used to direct therapeutic treatment or prophylaxis of these diseases and conditions.

Thus, according to the different aspects of the invention, there is provided methods for diagnosing higher susceptibility for various diseases and conditions in a human or animal, a new use of AKG and derivates, metabolites, analogues or salts thereof for the manufacture of a pharmaceutical preparation or a food or feed supplement for the in vivo therapeutic treatment or prophylaxis of various diseases, and methods of treatment or prophylaxis of various diseases and conditions in a human or animal.

In this specification, unless otherwise specified, “a” or “an” means “one or more”.

The terms “treatment or prophylaxis” in their various grammatical forms in relation to the present invention refer to preventing, curing, reversing, attenuating, alleviating, ameliorating, inhibiting, minimising, suppressing, or halting the disease/diseases or the deleterious effects of the disease/diseases associated with low levels of AKG in a human or animal.

According to the first aspect of the invention, a method is provided for diagnosing higher susceptibility for diseases and conditions associated with low levels of AKG in a human or animal comprising the following steps:

a) obtaining a biological sample from said human or animal;

b) measuring the AKG level in the biological sample; and

c) comparing said measured AKG level with normal average AKG levels, wherein a level of AKG in said sample lower than an average levels is indicative of a higher susceptibility for diseases and conditions associated with low levels of AKG.

Said diseases and conditions associated with low levels of AKG include: diseases or conditions associated with increased plasma levels of cholesterol, low density lipids and glycerides, such as arteriosclerosis, coronary disease, heart failure and cardiac death; diseases or conditions associated with bone loss or weakening, particularly osteoporosis; diseases or conditions associated with cartilage impairment and pain related to it, or arthrosis and rheumatoid arthritis and pain related to it; neoplastic diseases such as cancer and tumour growth; diseases of the nervous system, such as Alzheimer's, Parkinson, Huntington, Creutzweld-Jakob, BSE; renal failure; liver diseases such as liver cirrhosis, hepatitis or liver failure; heart conditions such as heart attack; stroke; bad wound healing; diseases or conditions related to infection of pathogenic strains of Helicobacter pylori, particularly diseases of the gastrointestinal tract including diseases such as gastritis, gastric and duodenal ulcers, peptic ulcer, gastric cancer, and gastric mucosa-associated lymphoid tissue lymphoma.

The term “test group”” in relation to the present invention refers to the group being diagnosed with the method according to the present invention.

The term “normal average AKG levels” in relation to the present invention refers to a determined baseline or mean level of AKG in biological samples, such as a blood samples, from a control group. The control group is preferably matched in relation to specific parameters such as sex, age, etc with the test group.

The determination of normal average AKG levels, which are used as comparison levels in the present method, is performed using standard methods of analysis well known in the art. This value will of course vary depending on for example kind of biological sample and group of subjects. Normal average blood levels of AKG in a group of humans are normally in the range of 5-7 μg/ml, but of course vary depending on age, sex, weight etc.

The term “low levels of AKG” in relation to the present invention refers to a level of AKG lower than the normal average AKG level of the control group.

Many different kinds of animals may be diagnosed with the method of the present invention, but a preferred animal for diagnosis is a human or a commercially valuable animal or livestock.

The step of obtaining a biological sample from said human or animal includes the process of being provided with a biological sample obtained with standard methods well known in the art. The biological sample could be, for example, blood, plasma, urine, vaginal secretions, tears, tissue, serum, stool, sputum, cerebrospinal fluid and supernatants from cell lysate. Preferably the biological sample is blood or urine.

The substance detected and measured in the method of the invention can be any naturally occurring form of AKG in the biological sample taken from a human or animal. This method includes measurement of alpha-ketoglutaric acid, alpha-ketoglutarate and other forms of AKG.

The step of measuring the AKG level in the biological sample is performed using standard detection techniques for measuring AKG in a sample well known in the art. Such techniques may include enzymatic methods and HPLC methods. However, it would be apparent to an artisan of skill in the art that these techniques are not the only suitable methods which may be used in the present invention.

Enzymatic methods for measuring AKG, also called “spectrometric methods” may, inter alia, comprise the following steps:

a) mixing a working solution comprising a buffer, ammonium ions, and NADH with a biological sample or a pre-treated form of a biological sample such as blood plasma collected from a centrifuged blood sample;

b) performing an initial spectrometric absorbance reading;

c) adding enzyme, preferably glutamate dehydrogenase; and

d) incubation to evoke a colour reaction; and

e) performing a second spectrometric reading of the colour reaction, after 10-minute incubation.

The amount of AKG in the sample is directly proportional to the decrease in absorbance between the first and second reading. The AKG concentration is calculated by the use of a standard curve.

A more detailed description of a specific enzymatic method for measuring AKG is described in Bergmeyer et al, 2-Oxoglutarate, UV spectrometric determination, in: methods of Enzymatic analyses, 2nd ed. (Bergmeyer H. U. ed.) Academic Press, New York, N.Y., 1974; and in Lambert, B. et al, Nutr. 132, 3383-3386, 2002.

HPLC methods for measuring AKG may, inter alia, comprise the following steps:

a) deproteinisation of a biological sample;

b) centrifugation of said deproteinised sample;

c) derivatization of the centrifuged sample; and

d) application of the derived sample on an HPLC (High Performance Liquid Chromatography) column.

A more detailed description of a specific HPLC method for measuring AKG is described in Kristensen, et al, Anim. Physiol. Anim. Nutr. 86, 1-7, 2002.

Further, other methods for measuring AKG are known in the art. For example, JP 04 349899 describes a method for measuring AKG in a specimen.

According to the second aspect of the invention, there is provided a use of a substance comprising at least one member selected from the group consisting of alpha-ketoglutaric acid (AKG) and derivates, metabolites, analogues or salts thereof for the manufacture of a pharmaceutical preparation or a food or feed supplement for the treatment of a human or animal with low levels of AKG compared with normal average AKG levels.

In a further embodiment, there is provided a use of a substance comprising at least one member selected from the group consisting of alpha-ketoglutaric acid (AKG) and derivates, metabolites, analogues or salts thereof for the manufacture of a pharmaceutical preparation or a food or feed supplement for the treatment or prophylaxis of diseases and conditions associated with low levels of AKG in a human or animal with low levels of AKG compared with normal average AKG levels.

Said diseases and conditions associated with low levels of AKG include: diseases or conditions associated with increased plasma levels of cholesterol, low density lipids and glycerides, such as arteriosclerosis, coronary disease, heart failure and cardiac death; diseases or conditions associated with bone loss or weakening, particularly osteoporosis; diseases or conditions associated with cartilage impairment and pain related to it, or arthrosis and rheumatoid arthritis and pain related to it; neoplastic diseases such as cancer and tumour growth; diseases of the nervous system, such as Alzheimer, Parkinson, Huntington, Creutzweld-Jakob, BSE; renal failure; liver diseases such as liver cirrhosis, hepatitis or liver failure; heart conditions such as heart attack; stroke; bad wound healing; diseases or conditions related to infection of pathogenic strains of Helicobacter pylori, particularly diseases of the gastrointestinal tract including diseases such as gastritis, gastric and duodenal ulcers, peptic ulcer, gastric cancer, and gastric mucosa-associated lymphoid tissue lymphoma.

It is known in the art that AKG can be used in a wide range of methods for treatment or prophylaxis of various diseases (see references below), but it has not yet been disclosed in the prior art to specifically treat humans or animals that have been diagnosed having low levels of AKG. WO 2005/123056 describes the use of AKG e.g. sodium alpha-ketoglutarate in the treatment of increased plasma levels of cholesterol, low density lipids and glycerides to lower the risk for of arteriosclerosis, coronary disease, heart failure and cardiac death. WO 03/043626 describes the use of AKG in a method for treating of a condition or conditions associated with bone loss or weakening, particularly osteoporosis. PCT/SE2006/050479 describes the use of AKG for the treatment, alleviation and prophylaxis of conditions associated with cartilage impairment and pain related to it, or prophylaxis of arthrosis and rheumatoid arthritis and pain related to it. WO 06/075924 describes the use of AKG in a method for treatment or prophylaxis of neoplastic diseases. WO 2006/016828 describes the use of AKG or its salt for augmenting, supporting functions of nerve cells and nervous system, and preventing e.g. Alzheimer's, Parkinson, Huntington, Creutzweld-Jakob, BSE. WO 03/055508 describes a composition useful for treating e.g. renal failure (acute and chronic renal failure, ACF and CRF) comprising AKG. Riedel, E. et al (Nephron, 74: pp 261-265, 1996) describes the administration of AKG to malnutritioned haemodialysis patients. DE 19929993 A1 describes the use of AKG in a method for treatment of liver diseases. Kjellman et al (Ann Thorac Surg, 63(6):1625-33 1997) describes that addition of alpha-ketoglutarate to blood improves cardioprotection during cardioplegia. Woollard et al (Stroke, 9(3):218-22, 1978) describes a controlled trial of ornithine alpha ketoglutarate (OAKG) in patients with stroke. Coudray-Lucas et al (Crit Care Med, 28 (6):1772-6, 2000) describes that ornithine alpha-ketoglutarate improves wound healing in severe burn patients. SE 0602446-7 describes the use of AKG for the in vivo therapeutic treatment or prophylaxis of diseases related to infection of pathogenic strains of Helicobacter pylori, particularly diseases of the gastrointestinal tract including diseases such as gastritis, gastric and duodenal ulcers, peptic ulcer, gastric cancer, and gastric mucosa-associated lymphoid tissue lymphoma. WO 2005/002567 describes the use of AKG in a method for improving absorption of amino acids as well as a method for decreasing absorption of glucose in a vertebrate. U.S. Pat. No. 5,646,187 describes the use of AKG in a method for treatment of critically ill patients for improving protein synthesis capacity, maintaining energy level, preserving the lean body mass and for improving the glutamine content in skeletal muscle.

The pharmaceutical preparation and the food or feed supplement of the present invention comprises at least one member selected from the group consisting of AKG and derivates, metabolites, analogues or salts thereof and mixtures thereof. Preferred are derivates, metabolites, analogues or salts of AKG, which produce the same or similar functions or therapeutic effects as AKG in its basic form.

The term “derivate” or “derivative” is herein intended to mean a chemical substance derived from AKG either directly or by modification or partial substitution. The term “metabolite” is herein intended to mean compounds that are break-down or degradation products of AKG. The term “analogue” or analog” is herein intended to mean compounds that are structurally similar to another, but are not necessarily isomers. Analogs have similar function(s) but differ in structure or evolutionary origin.

The pharmaceutically acceptable salt of alpha-ketoglutaric acid of the present invention could be any monovalent metal salt of alpha-ketoglutaric acid such as sodium, potassium salt or any divalent metal salt of alpha-ketoglutaric acid such as strontium, calcium or magnesium salt. As a preferred embodiment of the invention, alkali or alkaline earth metal salts of alpha-ketoglutaric acid are used, and most preferably sodium alpha-ketoglutarate is used.

Amino acids forming part of amides with alpha-ketoglutaric acid or of dipeptides with glutamine or glutamic acid or tripeptides with glutamine and/or glutamic acid may be any of the amino acids occurring as components in peptides in nature. The same applies to the pharmaceutically accepted physical mixtures of alpha-ketoglutaric acid or salts thereof with at least one amino acid. Preferably the amino acid or acids is/are selected from the group consisting of arginine, ornithine, leucine, isoleucine and lysine. Said amino acids are preferably used in their L-configuration.

Examples of amides of alpha-ketoglutaric acid with an amino acid or a di- or tripeptide include, but are not limited to, amides of alpha-ketoglutaric acid with an amino acid selected from the group consisting of glutamine, glutamic acid, arginine, ornithine, lysine, proline, isoleucine and leucine and amides of alpha-ketoglutaric acid with a dipeptide of glutamine and any of glutamic acid, arginine, ornithine, lysine, proline, isoleucine and leucine and with a dipeptide of glutamic acid and any of arginine, ornithine, lysine, proline, isoleucine and leucine.

Examples of di- and tripeptides of glutamine and glutamic acid with other amino acids include those mentioned above in connection with amides of alpha-ketoglutaric acid with di- or tripeptides.

Examples of physical mixtures of alpha ketoglutaric acid or salts thereof with at least one amino acid include, but are not limited to physical mixtures of at least one member selected from the group consisting of alpha-ketoglutaric acid and the sodium, potassium, calcium and magnesium salts thereof with any of glutamine, glutamic acid, arginine, ornithine, leucine, isoleucine, lysine and proline and any combinations of said amino acids.

A molar ratio of alpha-ketoglutaric acid or salts thereof to amino acid or amino acids of said physical mixtures will in general be within the limits of from 1:0.01 to 1:2, preferably from 1:0.1 to 1:1.5 and most preferably from 1:0.2 to 1:1.0.

The food or feed supplements and the pharmaceutical preparations of the present invention may be administered to a vertebrate, including mammals and birds, such as rodents (mouse, rat, guinea pig, or rabbit); birds (turkey, hen or chicken); other farm animals (cow, horse, pig or piglet); pets (dog, cat and other pets); and humans. While many animals may be treated with the preparation of the invention, a preferred animal for treatment is a human or commercially valuable animal and livestock.

Administration may be performed in different ways depending on what species of vertebrate to treat, on the condition of the vertebrate in the need of said treatment, and the specific indication to treat. The administration may be as a parenteral, rectal or oral food or feed supplement, as revealed above. Parenteral vehicles include sodium chloride solution, Ringer's dextrose, dextrose and sodium chloride, lactated Ringer's or fixed oils.

In one embodiment, the administration is done as a food or feed supplement, such as a dietary supplement and a component in form of solid food and beverage. Further embodiments may be in suspensions or solutions, such as a beverage further described below. Also, the formats may be in capsules or tablets, such as chewable or soluble, e.g. effervescent tablets, as well as powder and other dry formats known to the skilled man in the art, such as pellets, micro pellets, and grains.

The food and feed supplement may also be emulsified. The active therapeutic ingredient or ingredients may then be mixed with excipients, which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, buffering agents, which enhance the effectiveness of the active ingredient.

Different formats of the oral food or feed supplement may be supplied, such as solid food, liquids or lyophilised or otherwise dried formulations. It may include diluents of various buffers (e.g., Tris-HCl., acetate, phosphate), pH and ionic strength, additives such as albumin or gelatine to prevent absorption to surfaces, detergents (e.g., Tween 2 0, Tween 8 0, Pluronic F68, bile acid salts), solubilising agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the composition, complexation with metal ions, or in corporation of the material into or onto particulate preparations of polymeric compounds such as polylactic acid, polglycolic acid, hydrogels, etc. or onto liposomes, micro emulsions, micelles, unilamellar or multilamellar vesicles, erythrocyte ghosts, or spheroplasts. In one embodiment, the food or feed supplement is administered in the form of a beverage, or a dry composition thereof, in any of the methods according to the invention.

The beverage comprises an effective amount of the active ingredient or ingredients thereof, together with a nutritionally acceptable water-soluble carrier, such as minerals, vitamins, carbohydrates, fat and proteins. All of these components are supplied in a dried form if the beverage is provided in a dry form. A beverage provided ready for consumption further comprises water. The final beverage solution may also have a controlled tonicity and acidity, e.g. as a buffered solution according to the general suggestions in the paragraph above. The pH is preferably in the range of about 2-5, and in particularly about 2-4, to prevent bacterial and fungal growth. A sterilised beverage may also be used, with a pH of about 6-8. The beverage may be supplied alone or in combination with one or more therapeutically effective composition.

According to a further embodiment, the pharmaceutical preparations of the invention for oral use may be in the form of tablets, lozenges, capsules, powders, aqueous or oily suspensions, syrups, elixirs, aqueous solutions and the like comprising the active ingredient or ingredients in admixture with a pharmaceutically acceptable carrier and additives, such as diluents, preservatives, solubilisers, emulsifiers, adjuvants and carriers useful in the methods and use disclosed in the present invention.

Orally applied alpha-ketoglutaric acids²⁻ in form of salts can be absorbed to the gut and stomach epithelia (Kristensen, N B., et al, J. Anim. Physiol. Anim. Nutr. 86, 1-7, 2002; Lambert, B., et al, J. Nutr. 132, 3383-3386, 2002) via specific co-transporters (Buddington, R. K., et al, Comparative Biochemistry and Physiology, Part A.138/2, 215-220, 2004).

Further, as used herein “pharmaceutically acceptable carriers” are well known to those skilled in the art and may include, but are not limited to, 0.01-0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/-aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

The term “therapeutically effective amount” in relation to the present invention refers to that amount which provides a therapeutic effect for a given condition and administration regimen. This is a predetermined quantity of active material calculated to produce a desired therapeutic effect in association with the required additives and diluents; i.e., a carrier, or administration vehicle. Further, it is intended to mean an amount sufficient to reduce and most preferably prevent a clinically significant deficit in the activity and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluents; i.e., carrier, or additive. Further, the dosage to be administered will vary depending on the active principle or principles to be used, the condition to be treated, the age, sex, weight etc. of the patient to be treated but will generally be within the range from 1 to 1000 mg/kg body weight/day, or from 10 to 400 mg/kg body weight and day, preferably from 10 to 100 mg/kg body weight/day.

### BRIEF DESCRIPTION OF THE DRAWINGS

FIG. 1 shows the disease frequency in % of various diseases and conditions in relation to AKG blood level of patients examined in Example 2.

### EXAMPLES

The following examples are given to illustrate the present invention. It should be understood, however, that the scope of the invention is not intended to be limited to the specific conditions and details described in these examples, but should only be limited by the wording of the claims.

### Example 1

### Effect of Dietary AKG on Surviving of Old Rats

The aim of this experiment was to study the relationship between the blood level of AKG and survival of elderly rats. Forty Sprague-Dawley rats randomly chosen (2-3 years old) from the Department of Cell & Organisms Biology in Lund University were used in the experiment. The rats were divided into four groups, 10 rats each, two control groups and two experiment groups. Control Group I was given standard food, H₂O, NaCl (1.17 g), glucose and saccharose; Control Group II was given standard food, H₂O, NaCl (11.7 g), glucose and saccharose; Experiment Group I was given standard food, H₂O, Na₂AKG * 2H₂O (2.28 g), glucose and saccharose; and Experiment Group II was given standard food, H₂O, Na₂AKG * 2H₂O (22.8 g), glucose and saccharose. The different ingredients and the amounts of these and the pH are shown in Table 1. The mixtures were titrated with 0.01 M NaOH to correct the pH.

TABLE 1

| | | | | | | | Experiment | | | Experiment | | |
|---|---|---|---|---|---|---|---|---|---|---|---|---|
| | Control Group I | | | Control Group II | | | Group I | | | Group II | | |
| Ingredients | 11 | 21 | 41 | 11 | 21 | 41 | 11 | 21 | 41 | 11 | 21 | 41 |
| Na₂AKG * | — | — | — | — | — | — | 2.28 | 4.56 | 9.12 | 22.8 | 45.6 | 91.2 |
| 2H₂O | | | | | | | | | | | | |
| NaCl | 1.17 | 2.34 | 4.68 | 11.7 | 23.4 | 46.8 | — | — | — | — | — | — |
| C₆H₁₂O₆ | 30 | 60 | 120 | 30 | 60 | 120 | 30 | 60 | 120 | 30 | 60 | 120 |
| Glucose | | | | | | | | | | | | |
| C₁₂H₂₂O₁₁ | 15 | 30 | 60 | 15 | 30 | 60 | 15 | 30 | 60 | 15 | 30 | 60 |
| Sucrose | | | | | | | | | | | | |

| | | | | |
|---|---|---|---|---|
| pH | 7.35-7.38 | 7.35-7.38 | 7.35-7.38 | 7.35-7.38 |

Blood samples were taken at the beginning of the experiment from all rats and blood AKG levels were estimated. Experimental time was 8 weeks and parameters as AKG blood levels and survival were monitored.

### Results

Table 2 shows the blood AKG levels and survival of the test rats in the 10 beginning and in the end of the experiment. In the table the different average results describe statistical differences when p<0.05.

TABLE 2

| | Number of dead | AKG μg/ml | AKG μg/ml |
|---|---|---|---|
| Groups | animals | Day 1 | Day 56 |
| Control Group I | 3 | <0.1 | - died |
| | | 0.38 | 0.42 |
| | | 0.1 | - died |
| | | 0.8 | 0.7 |
| | | 0.3 | 0.3 |
| | | <0.1 | - died |
| | | 0.42 | 0.4 |
| | | 0.56 | 0.52 |
| | | 0.37 | 0.38 |
| | | 0.2 | 0.15 |
| Average AKG blood | | 0.30 ± 0.19 | 0.41 ± 0.17 |
| level before and after | | | |
| the experiment | | | |
| Control Group II | 2 | 0.31 | 0.34 |
| | | 0.52 | 0.62 |
| | | <0.1 | - died |
| | | <0.1 | - died |
| | | 0.36 | 0.35 |
| | | 0.33 | 0.24 |
| | | 0.15 | 0.25 |
| | | 0.45 | 0.5 |
| | | 0.27 | 0.25 |
| | | 0.54 | 0.2 |
| Average AKG blood | | 0.31 ± 0.16 | 0.34 ± 0.14 |
| level before and after | | | |
| the experiment | | | |
| Experiment Group I | 0 | 0.34 | 0.95 |
| | | 0.31 | 0.75 |
| | | <0.1 | 0.82 |
| | | 0.2 | 0.78 |
| | | 0.54 | 1.04 |
| | | 0.32 | 0.9 |
| | | 0.15 | 0.7 |
| | | 0.23 | 0.6 |
| | | 0.15 | 0.6 |
| | | 0.6 | 0.9 |
| Average AKG blood | | 0.29 ± 0.16 | 0.80 ± 0.14 |
| level before and after | | | |
| the experiment | | | |
| Experiment Group II | 0 | 0.34 | 1.5 |
| | | 0.24 | 0.98 |
| | | 0.23 | 0.89 |
| | | <0.1 | 0.7 |
| | | 0.75 | 1.4 |
| | | 0.34 | 1.11 |
| | | 0.5 | 1.07 |
| | | 0.42 | 0.95 |
| | | 0.25 | 0.97 |
| | | 015 | 0.95 |
| Average AKG blood | | 0.33 ± 0.18 | 1.05 ± 0.23 |
| level before and after | | | |
| the experiment | | | |

This study on elderly rats (over 24 months) shows that rats with a blood AKG level under 0.1 μg/ml have a predisposition for sudden death. Oral administration of AKG can restore blood level of AKG to the level around 1 μg/ml. The survival rate of the rats in the experimental groups drastically improved. Their blood levels of AKG were elevated over 0.5 μg/ml after being orally feed with AKG sodium salt, and rats with initially low levels, under 0.1 μg/ml, of blood AKG, did survive the whole experiment. Hence, it has been shown that it can be of diagnostic value to monitor the AKG blood level, and that administration of AKG to elevate low levels of AKG in blood can have a therapeutic value.

### Example 2

### AKG Blood Level and its Relation to Various Diseases

The aim of this experiment was to study the relationship between AKG blood levels and various diseases in different age groups in human. Hence, the end point of this study was to measure AKG blood levels and relate it to health status e.g., cholesterol levels (LDL), osteoporosis, arthritis, cancer, neural disorders, kidney function, liver function, heart attack , stroke, wound healing, gastritis related to H. pylori colonisation, and other systemic diseases.

The study was designed as a randomized, double blind, 1 month study—conducted at the General Medicine Outpatient Department of the Institute of Agricultural Medicine (IAM) in Lublin, Poland. The study population was randomly assigned to measure blood levels of AKG during first visit.

Patients were recruited from whole population seeking first visit to the General Medicine Department of the Institute of Agricultural Medicine. 23 patients were screened for the study. Of these, 11 patients were women and 12 men, aged 12-88 years. The protocol was approved by the Institute's Review Board and local Ethics Committee. All patients gave their informed consent.

Plasma AKG was determined by the method of Bergmeyer and Bernt (Bergmeyer et al, 2-Oxoglutarate, UV spectrometric determination, in: methods of Enzymatic analyses, 2nd ed. (Bergmeyer H. U. ed.) Academic Press, New York, N.Y., 1974;) with minor modifications. The assay was carried out in 0.5 ml of working solution consisting of 100 mmol/l phosphate buffer (pH 7.6), 4 mmol/l ammonium chloride, and 50 μmol/l NADH. To the working solution, an appropriate amount of plasma containing 1-10 nmol of AKG was added. An initial absorbance reading was obtained at 340 nm. Following the initial absorbance reading, ˜6 units (in a volume of 10 μL) of bovine GDH (G2501; Sigma-Aldrich, St. Louis, Mo.) was added to each tube. After a 10-minute incubation, a second absorbance reading was taken at 340 nm. The amount of AKG in the sample is directly proportional to the decrease in absorbance between the first and second reading. The AKG concentration was calculated by the use of a standard curve.

The level of AKG in blood does not show any substantial variation during the 24 hour cycle.

All patients were examined by the same doctor, illnesses were diagnosed by accordingly basic physical examination, vital signs, haematology, biochemistry, and basic urine analysis for the day of study visits.

Severity of the sickness was evaluated in 4-stage scale: (−) no signs of sickness; (+) mild signs of sickness; (++) moderate sign of sickness; and (+++) severe sign of sickness.

### Results

No adverse events were recorded at the visits after blood sampling. Table 3 shows the relation between the frequency of different diseases and AKG levels in blood. The following diseases and conditions were estimated: cholesterol/LDL levels (LD), osteoporosis (Os), arthritis (Ar), cancer (Ca), neural disorders (Ne), kidney failure (Ki), liver dysfunction (Li), heart attack (Ha), stroke (Str), wound healing (W), H. pylori related Gastritis (HP), and other diseases—not related to AKG.

TABLE 3

| | | | | | | | | | | | | | | Other |
|---|---|---|---|---|---|---|---|---|---|---|---|---|---|---|
| Patient | Age | AKG ug/ml | LD | Os | Ar | Ca | Ne | Ki | Li | Ha | Str | W | HP | dis. |
| Male | 65 | <1 | +++ | + | ++ | − | − | + | + | + | − | + | + | − |
| Fem. | 56 | 3.3 | − | − | − | − | − | − | + | − | − | − | − | + |
| Fem. | 51 | <1 | ++ | + | + | + | + | + | + | + | − | + | + | + |
| Male | 25 | 5 | − | − | − | − | − | − | + | − | − | − | − | + |
| Male | 32 | 1.3 | ++ | − | ++ | + | + | − | − | − | − | + | + | − |
| Male | 71 | 2.7 | − | − | − | − | − | − | − | − | + | − | − | + |
| Male | 66 | <1 | +++ | + | + | + | + | + | − | + | + | + | − | − |
| Fem. | 44 | 1 | ++ | ++ | + | + | + | − | − | − | − | + | + | − |
| Fem. | 51 | 2.7 | − | − | − | + | − | − | − | − | − | − | − | + |
| Fem. | 15 | 6.5 | − | − | − | − | − | − | − | − | − | − | − | + |
| Male | 23 | 2.5 | + | − | − | − | − | − | + | − | − | − | + | − |
| Male | 69 | 2 | − | − | + | − | − | − | − | − | − | − | − | + |
| Fem. | 51 | 1 | ++ | ++ | + | + | − | + | − | − | − | − | + | − |
| Fem. | 53 | 2.3 | − | − | − | − | − | − | + | + | − | − | − | |
| Fem. | 49 | 3.5 | − | − | − | − | − | − | − | − | − | − | − | + |
| Fem. | 21 | 6.7 | − | − | − | − | − | − | − | − | − | − | − | + |
| Male | 18 | 7.1 | − | − | − | − | − | − | − | − | − | − | − | + |
| Male | 13 | 9.1 | − | − | − | − | − | − | − | − | − | − | − | + |
| Male | 88 | 3.9 | − | − | − | − | − | − | − | − | − | − | − | + |
| Fem. | 54 | 4.1 | | − | − | − | − | − | − | − | − | − | − | + |
| Male | 42 | 2.5 | + | − | + | + | − | − | − | − | − | − | + | − |
| Male | 45 | 2.0 | + | − | + | + | − | − | + | + | − | − | + | − |
| Fem. | 16 | 2.5 | + | − | + | + | − | − | − | + | − | − | + | − |

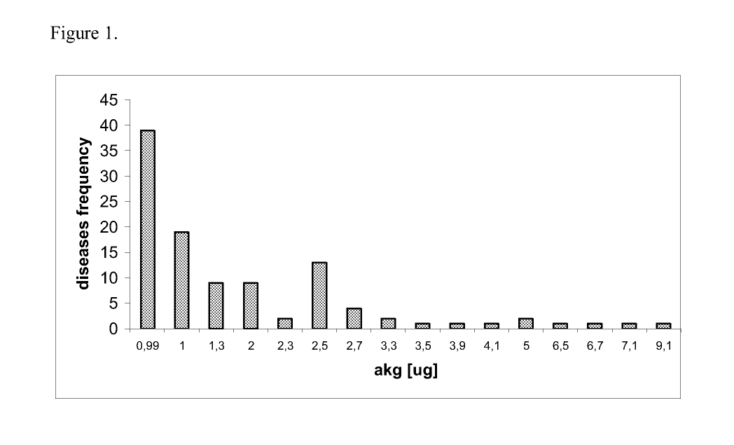

FIG. 1. shows the disease frequency in % of the various diseases and conditions in relation to AKG blood level.

For the statistical analysis the patients were divided into the following groups considering their blood levels of AKG: <2 μg/ml; 2-5 μg/ml; and >5 μg/ml. Chi-square indicates that all groups are different considering frequency of the diseases and conditions on the level p<0.000.

This study tested humans of different sex and age towards correlation between blood AKG levels and their susceptibility for different diseases. It shows that humans with low blood levels of AKG have predisposition for various diseases and conditions. A blood AKG level under 2.5 μg/ml independently of age is correlated in patients with several disorders such as H. pylori related gastritis, heart disorders, elevated level of LDL, arthritis, and a blood level of AKG under 1 μg/ml additionally is correlated with osteoporosis, cancer and bad wound healing. Hence, it has been shown that it can be of diagnostic value to monitor the AKG blood level in humans, and that administration of AKG to elevate low levels of AKG in blood can have a therapeutic value.

1-28. (canceled)

29. A method for detecting higher susceptibility for a first group of disorders in humans or animals, the first group of disorders comprising the following disorders:
increased plasma levels of cholesterol, low density lipids and glycerides;
arthritis, diseases or conditions associated with cartilage impairment;
neural disorders, diseases of the nervous system;
renal failure;
liver dysfunction;
heart attack, heart disorders;
stroke; and
Helicobacter pylori related diseases or conditions of the gastrointestinal tract, the method comprising:
obtaining a blood sample from said human or animal;
measuring alpha-ketoglutaric acid (AKG) plasma level in the blood sample; and
evaluating said measured AKG level wherein a blood plasma AKG level under 2.5 microgram per millilitre indicates a higher susceptibility for the first group of disorders.

30. The method of claim 29, additionally for detecting higher susceptibility for a second group of disorders in humans, the second group of disorders comprising the following disorders:
the first group of disorders;
osteoporosis;
cancer; and
bad wound healing;
the method comprising:
obtaining a blood sample from said human or animal;
measuring alpha-ketoglutaric acid (AKG) plasma level in the blood sample; and
evaluating said measured AKG level wherein a blood plasma AKG level under 1.0 microgram per millilitre indicates a higher susceptibility for the second group of disorders.

31. A method for detecting susceptibility to disease, comprising:
obtaining a blood sample from a human or animal;
measuring alpha-ketoglutaric acid (AKG) level in the blood sample; and
evaluating said measured AKG level wherein a blood AKG level of <0.1 μg/ml indicates a high risk for death in near term, 0.1-2 μg/ml indicates susceptibility to a serious disease condition, 2-5 μg/m1 indicates susceptibility to a moderately serious disease condition and >5 μg/ml indicates no particular susceptibility to disease.

32. The method according to claim 31, wherein said disease or susceptibility to disease to be detected specifically includes one or more of the following conditions: diseases or conditions associated with increased plasma levels of cholesterol, low density lipids and glycerides; diseases or conditions associated with bone loss or weakening; diseases or conditions associated with cartilage impairment; neoplastic diseases; diseases of the nervous system; renal failure; liver diseases; heart conditions; stroke; bad wound healing; and Helicobacter pylori related diseases or conditions of the gastrointestinal tract.

33. The method according to claim 31, wherein said disease or susceptibility to disease to be detected specifically includes one or more of the following conditions: osteoporosis, arthrosis, cancer, renal failure, liver dysfunction, heart attack, stroke, bad wound healing, Helicobacter pylori related gastritis.

34. The method according to claim 31, wherein said disease or susceptibility to disease to be detected specifically includes one or more of the following conditions: gastric and duodenal ulcers, peptic ulcer, gastric cancer, gastric mucosa-associated lymphoid tissue lymphoma, arteriosclerosis, coronary disease, heart failure, cardiac death, rheumatoid arthritis, liver cirrhosis, hepatitis and liver failure

35. The method according to claim 29, wherein a blood level of <1 μg/ml is indicative of the presence of one or more of the following diseases: osteoporosis, cancer, bad wound healing, Helicobacter pylori related gastritis, heart disorders, elevated level of LDL and arthritis.

36. The method according to claim 29, wherein a blood AKG level of 1-2.5 μg/ml is indicative of the presence of one or more of the following diseases: Helicobacter pylori related gastritis, heart disorders, elevated level of LDL and arthritis.

37. A method for prophylaxis of a disease in a human or animal subject having a blood AKG level of <5 μg/ml, which method comprises administering to a subject in need of such prophylaxis an effective amount of at least one member selected from the group consisting of alpha-ketoglutaric acid (AKG) and derivates, metabolites, analogues or salts thereof

38. The method according to claim 37 wherein the subject has a blood AKG level of <1 μg/ml.

39. The method according to claim 38 wherein the subject has a blood AKG level of <0.1 μg/ml.

40. The method according to claim 37, wherein the subject is elderly.

41. The method according to claim 37, wherein the subject is suffering from one or more of the following: diseases or conditions associated with increased plasma levels of cholesterol, low density lipids and glycerides; diseases or conditions associated with bone loss or weakening;
diseases or conditions associated with cartilage impairment; neoplastic diseases; diseases of the nervous system; renal failure; liver diseases; heart conditions; stroke; bad wound healing; and
Helicobacter pylori related diseases or conditions of the gastrointestinal tract.

42. The method according to claim 37, wherein the subject is suffering from one or more of the following: arteriosclerosis, coronary disease, heart failure and cardiac death; osteoporosis;
arthrosis and rheumatoid arthritis; neoplastic diseases and cancer; renal failure; liver cirrhosis, hepatitis or liver failure; heart attack; stroke; bad wound healing; Helicobacter pylori related gastritis, gastric and duodenal ulcers, peptic ulcer, gastric cancer, and gastric mucosa-associated lymphoid tissue lymphoma.

43. The method according to claim 37, wherein said at least one member is selected from the group consisting of AKG, glutamine, glutamic acid and pharmaceutically acceptable salts of these acids, amides of AKG and an amino acid or a di- or tripeptide, di- or tripeptides of glutamine and other amino acids, di- or tripeptides of glutamic acid and other amino acids, pharmaceutically acceptable salts of said di- or tripeptides, and pharmaceutically accepted physical mixtures of AKG or a pharmaceutically acceptable salt thereof and at least one amino acid.

44. The method according to claim 37, wherein said at least one member is selected from the group consisting of ornithine-AKG, arginine-AKG, glutamine-AKG, glutamate-AKG, leucine-AKG, chitosan-AKG, or mixtures thereof.

45. The method according to claim 37, wherein said at least one member is selected from the group consisting of an alkali or alkaline earth metal salt of AKG, or mixtures thereof.

46. The method according to claim 37, wherein the amount of AKG given to a human or animal is a therapeutically effective amount.

47. The method according to claim 46, wherein the therapeutically effective amount is in the interval from 1 to 1000 mg/kg body weight/day.

48. The method according to claim 47, wherein the therapeutically effective amount is in the interval from 10 to 400 mg/kg body weight/day.

49. The method according to claim 48, wherein the therapeutically effective amount is in the interval from 10 to 100 mg/kg body weight/day.

50. A method of increasing quality of life comprising the following steps: measuring the alpha-ketoglutaric acid (AKG) level in a biological sample; supplementing low levels of AKG with AKG to normalize said AKG levels.

51. A method to treat a first group of disorders in a subject comprising administering to a subject in need thereof an effective amount of at least one member selected from the group consisting of alpha-ketoglutaric acid (AKG) and derivates, metabolites, analogues or salts thereof
wherein said subject suffers from a first group of disorders comprising the following disorders:
increased plasma levels of cholesterol, low density lipids and glycerides; ;
arthritis, diseases or conditions associated with cartilage impairment;
neural disorders, diseases of the nervous system;
renal failure;
liver dysfunction;
heart attack, heart disorders;
stroke; and
Helicobacter pylori related diseases or conditions of the gastrointestinal tract, and wherein alpha-ketoglutaric acid (AKG) plasma level in a blood sample from said subject is under 2.5 microgram per millilitre.

52. The method of claim 51, further comprising a second group of disorders, the second group comprising the disorders of the first group and additionally the following disorders:
osteoporosis;
cancer; and
bad wound healing;
wherein the alpha-ketoglutaric acid (AKG) plasma level in a blood sample from the subject is under 1.0 microgram per millilitre.
